(19) United States
(12) Patent Application Publication
(10) Pub. No.: US 2003/0097073 A1
(43) Pub. Date: May 22, 2003

(54) Pressure cuff

(75) Inventors: Edward Theodore Bullister, Weston, MA (US); James E. Sluetz, North Attleboro, MA (US); Sanford Reich, Providence, RI (US); Neil Silverman, Framingham, MA (US)

Correspondence Address: FRANCIS L CONTE, 6 PURITAN AVENUE, SWAMPSCOTT, MA, 01907

(73) Assignee: APEX Medical, Inc., East Walpole, MA

(21) Appl. No.: 10/001,836

(22) Filed: Nov. 16, 2001

(51) Int. Cl. A61B 5/02

(52) U.S. Cl. 600/486

(57) ABSTRACT

A pressure cuff includes a pad having a seat for receiving a vessel therealong through which a fluid under pressure flows. A pressure sensor is mounted inside the pad and includes a flexible flat diaphragm in the seat for engaging the vessel in flat contact therewith. A flexible strap is provided to clamp the pad in abutting contact with the vessel for measuring pressure through the wall thereof.

### BACKGROUND OF THE INVENTION

The present invention relates generally to pressure sensors, and, more specifically, to implantable pressure sensors.

Living bodies include various vessels containing fluid under pressure. Exemplary vessels include arteries, veins, lumens, vesicles, tubes, ducts, as well as arterial grafts.

In a particular example, a Left Ventricular Assist Device (LVAD) is implanted in a living body to assist the pumping function of the heart therein. The LVAD is attached to the heart using a suitable graft, and it is desirable to measure pressure of blood discharged through the LVAD for use and control thereof.

Chronic or long term pressure measurement is desired in both animal and human medical studies, as well as for various forms of medical treatment including the temporary use of the LVAD. Since this critical application of pressure measurement includes living cells, pressure measurement must be effected with relatively little or no damage to those cells.

Accordingly, it is desired to provide a pressure sensor specifically configured for use in living bodies for chronic measurement of fluid pressure with little or no trauma to living cells.

### BRIEF SUMMARY OF THE INVENTION

A pressure cuff includes a pad having a seat for receiving a vessel therealong through which a fluid under pressure flows. A pressure sensor is mounted inside the pad and includes a flexible flat diaphragm in the seat for engaging the vessel in flat contact therewith. A flexible strap is provided to clamp the pad in abutting contact with the vessel for measuring pressure through the wall thereof.

### BRIEF DESCRIPTION OF THE DRAWINGS

The invention, in accordance with preferred and exemplary embodiments, together with further objects and advantages thereof, is more particularly described in the following detailed description taken in conjunction with the accompanying drawings in which:

FIG. 1 is a top plan view of a pressure cuff mounted around a fluid carrying vessel in accordance with an exemplary embodiment of the present invention.

FIG. 2 is a partly sectional side view of the pressure cuff illustrated in FIG. 1 and taken along line 2-2.

FIG. 3 is a radial sectional view through the pressure cuff illustrated in FIG. 1 and taken along line 3-3.

FIG. 4 is a partly sectional side view of the pressure cuff illustrated in FIG. 1 and taken along line 4-4.

FIG. 5 is an isometric view of a flexible strap for the pressure cuff illustrated in FIG. 4 in accordance with another embodiment of the present invention.

FIG. 6 is a top view of a portion of the pressure cuff illustrated in FIG. 1 having a strap clamp in accordance with another embodiment of the present invention.

FIG. 7 is an isometric view of a pressure cuff having a strap clamped in accordance with another embodiment of the present invention.

FIG. 8 is a elevational sectional view through a portion of the pressure cuff illustrated in FIG. 7 and taken along line 8-8.

### DETAILED DESCRIPTION OF THE INVENTION

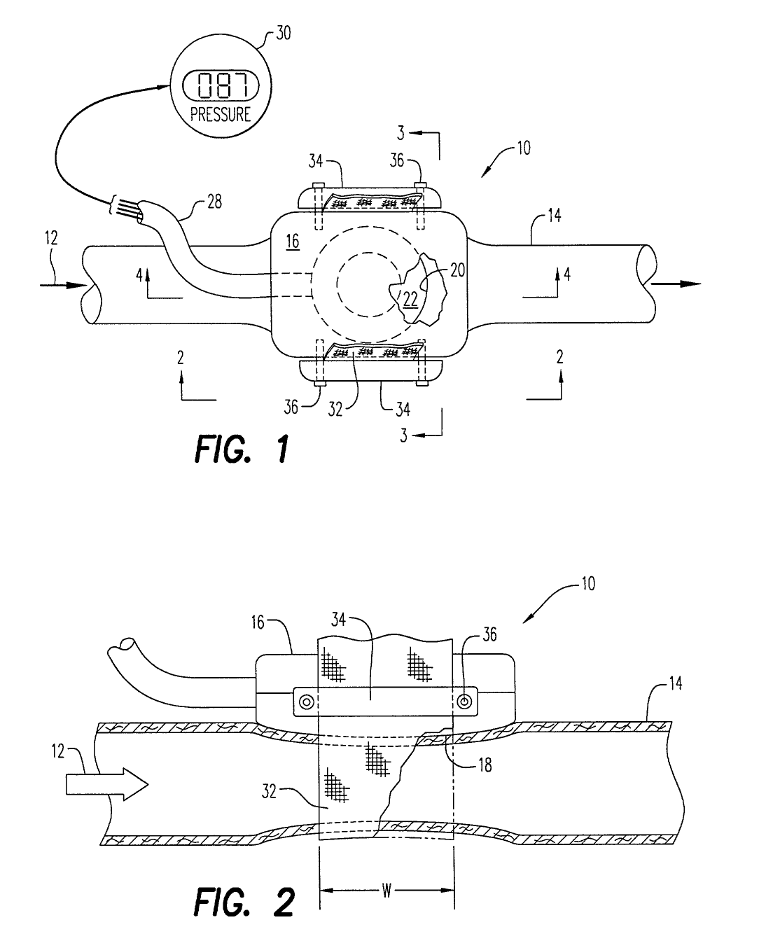

Illustrated in FIG. 1 is a pressure monitor or cuff 10 specifically configured for measuring pressure of a fluid 12 carried through or contained in a flexible vessel 14 in accordance with an exemplary embodiment. The vessel 14 may have any flexible form in which pressure measurement of fluid contained therein is desired, and in the example illustrated in FIG. 1 is found in a living body, either animal or human. Various animal or human medical studies, or medical treatment, may use to advantage chronic measurement of fluid pressure inside the vessel with little or no damage to living cells.

In the exemplary embodiment illustrated in FIG. 1, the vessel 14 is an artery or arterial graft through which flows the fluid 12 in the form of blood. A typical example is the discharge of blood through the vessel 14 from the LVAD assisting a natural heart in the example introduced above.

Accordingly, it is desired to externally measure blood pressure inside the vessel 14 without breaching the arterial wall thereof, or causing undesirable trauma thereto. The pressure cuff 10 is specifically configured to engage the flexible vessel for detecting pressure therein, and may be formed of any suitable biocompatible materials for long term implantation into the body.

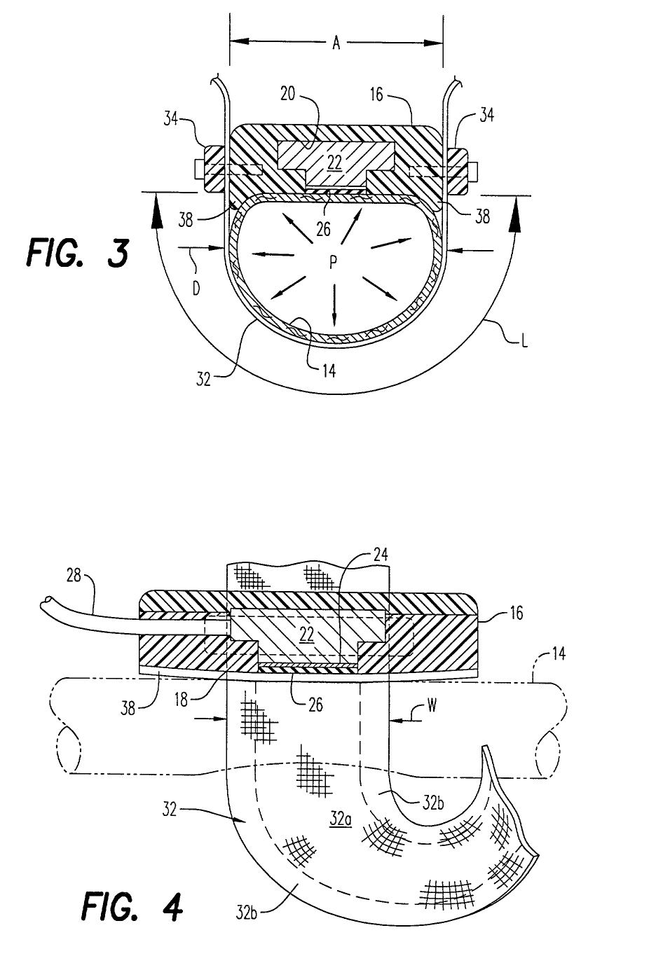

The pressure cuff is illustrated in top view in FIG. 1, side view in FIG. 2, and in radial, transverse sectional view in FIG. 3. The cuff includes a rigid foundation or pad 16 which may be formed of any suitable biocompatible material such as plastic or metal that may be coated as desired. The pad is generally a rectangular block having a longitudinal seat 18 extending along the bottom side thereof for receiving the vessel 14 axially along the longitudinal axis thereof. As shown in FIGS. 1 and 3, the pad includes an internal pocket or well 20 specifically configured for mounting therein a pressure sensor 22.

As shown in side sectional view in FIG. 4, the pressure sensor 22 preferably includes a flexible flat diaphragm 24 which deflects under pressure, and which deflection is measured for correspondingly measuring the pressure applied thereto. Flat diaphragm pressure sensors are conventional and may be sized with sufficiently small detection diaphragms for use in the pressure cuff. One type of suitable pressure sensor is known as a Micro-Electro-Mechanical-System (MEMS) sensor which is mass produced from silicon wafers and has large pressure sensitivity. Manufacturers of such diaphragm pressure sensors include Druck Incorporated of New Fairfield, Conn.; and Kulite Semiconductor Products, Inc. of Leonia, N.J.

The main diaphragm 24 isolates the pressure sensor from the environment and is typically metal. In the preferred embodiment illustrated in FIG. 4, the main metal diaphragm 24 is covered by a secondary flexible flat diaphragm 26 which is preferably elastomeric, such as silicone, and is formed flush or coextensive with the surrounding seat 18.

The seat and exposed diaphragm 26 have a smooth surface finish to protect the vessel 14. The exposed diaphragm 26 provides direct contact with the surface of the vessel for transferring pressure forces therefrom to the main diaphragm 24 which undergoes flexing under pressure, which flexing is suitably detected by the operative elements of the pressure sensor for producing a pressure signal. An additional advantage of the silicone diaphragm 26 is its ability to frictionally engage the vessel for maintaining position of the pad thereon without unacceptable slipping therealong.

The pressure sensor 22 is preferably hermetically sealed within its supporting pad 16 and includes an electrical lead 28 extending therefrom and joined to a suitable pressure indicator 30 illustrated in FIG. 1 for displaying the pressure measured inside the vessel. The indicator may be configured for visual observation, or may simply form part of an electric circuit in which the pressure signal is used, such as in controlling operation of the LVAD.

The pressure sensor and its cooperating pressure indicator are suitably electrically powered in any convenient manner. Although the pressure sensor may be hard wired to the pressure indicator, suitable telemetry may be used for transmitting the pressure signals from the sensor to the indicator for permitting the complete implantation of the pressure sensor inside a living body without the need for the retention of access openings through the body for communication with the pressure sensor.

As shown in FIGS. 3 and 4, the diaphragm 26 is preferably disposed centrally in the bottom seat 18 of the pad flush with the surface thereof for correspondingly engaging the vessel 14 in flat contact therewith. Since the vessel is flexible, it readily complies or conforms with the shape of the seat and the flat diaphragm 26 for transmitting the pressure (P) of the fluid to the diaphragm for measurement thereof as illustrated in FIG. 3.

However, in view of the flexibility of the vessel 14, the pressure cuff also includes a flexible band or strap 32, as shown in FIGS. 1-4, which is suitably adjustably joined to the pad for clamping the pad and seat in flat abutting contact with the vessel. The strap 32 is sufficiently flexible to conform to the distorted shape of the vessel as the vessel is clamped in flat abutment against the seat 18 as illustrated in FIG. 3.

As initially illustrated in FIG. 1, suitable means in the exemplary form of one or more clamps 34 are provided for adjustably joining opposite ends of the flexible strap 32 to the rigid pad 16. As shown in FIG. 3, the strap is sufficiently long to define a strap loop around the bottom side of the vessel with a corresponding length L between the opposite clamps 34 to entrap the vessel 14 against the pad seat.

Correspondingly, the top side of the vessel is bent or compressed flat against the diaphragm 26 and conforms with the shape of the pad seat. In this way, the flexible vessel is compressed between the pad and strap and is free to conform to the resulting configuration thereof which is generally D-shaped in section as illustrated in FIG. 3. The conforming flexibility of the strap reduces the likelihood of trauma or damage to the flexible vessel as it is distorted to engage the flat diaphragm.

As shown in FIGS. 1 and 2, each clamp 34 is preferably a straight bar straddling the opposite ends of the strap against a suitable portion of the pad, such as its opposite sides. A pair of fasteners 36 such as screws are disposed at opposite ends of each bar and threadingly engage the pad.

In this way, the fasteners may be adjusted to permit the sliding movement of the opposite ends of the strap between the corresponding clamps and the pad for initial assembly of the pressure cuff around the intended pressure vessel 14. The strap is suitably threaded through the two clamp slots for compressing the pad against the vessel. The fasteners may then be tightened to secure the cuff around the intended location on the vessel.

FIGS. 1-3 illustrate the final assembly of the pressure cuff around the flexible vessel 14 in various views in which the nominally round or tubular vessel is locally distorted under the retention forces of the pressure cuff. In order to protect the vessel from undesirable damage, the strap 32 has a suitable width W to spread the retention forces of the strap over a suitable portion of the length of the vessel. For example, for the arterial graft illustrated, the strap width W may be about 25 mm.

The strap 32 is preferably axially wider in width W than long in length L between the pad curbs 38. The inter-curb length L is selected for entrapping the corresponding diameter of the intended vessel. And the larger strap width ensures that the entrapping forces from the pressure cuff are suitably spread axially along a suitable portion of the vessel for minimizing the likelihood of damage or trauma thereto.

FIG. 3 illustrates that the vessel is distorted in its radial direction when clamped inside the pressure cuff. And, FIGS. 1 and 2 illustrate that the vessel is also distorted along its length in the local vicinity of the applied pressure cuff. As shown in FIG. 2, the seat 18 is preferably axially or longitudinally flared convex between the opposite axial ends of the pad 16 for protecting the flexible vessel as it is distorted under the clamping forces.

The seat 18 is generally straight at its middle in which the pressure measuring diaphragm is located, but is generally convex outwardly at opposite ends of the pad. In this way, the flexible vessel 14 as illustrated in FIG. 2 may gradually change from its nominally circular profile to the generally D-shaped profile illustrated in FIG. 3 at the pressure-measuring diaphragm 26. As shown in FIG. 2, no sharp edges are provided on the opposite ends of the pad to prevent local stress concentrations against the wall of the vessel.

As shown in FIG. 3, the seat 18 is also circumferentially flared concave between opposite circumferential ends or curbs 38 of the pad 16. As indicated above, for accurate pressure measurement the exposed diaphragm 26 is flat, and the corresponding portion of the flexible vessel 14 is distorted from its nominal arcuate shape to a flat shape abutting the diaphragm 26. The opposite portion of the vessel 14 constrained by the flexible strap 32 remains arcuate, and requires a suitable transition and shape where it joins the rigid pad at opposite ends thereof. The arcuate or generally concave outwardly curbs 38 provide a smooth transition from the flat portion of the constrained vessel to its arcuate portion retained by the strap to minimize any damage in these transition regions.

The pad 16 illustrated in FIG. 3 may be formed as small as practical for supporting therein the pressure sensor 22 with its pressure-sensing flat diaphragm. In the preferred embodiment illustrated in this Figure, the pad 16 is sized in width A to cover or surround a minority of the circumference of the flexible vessel 14, and correspondingly the strap 32 is sized with sufficient length L between the two clamps to surround the remaining majority of the circumference of the vessel.

The pad seat 18 illustrated in FIG. 3 preferably matches the pad width A which is generally equal to the outer diameter D of the strap loop when conforming to the entrapped vessel 14 therein. In this way, the opposite ends of the strap extend generally vertically through the clamps and generally tangential to the opposite sides of the entrapped vessel.

Correspondingly, the pad seat 18 is typically larger than the supported diaphragms and is preferably flat in the transverse or circumferential direction between the arcuate curbs 38. However, the seat may be circumferentially arcuate outboard of the flat diaphragm for reducing the distorted shape of the vessel when captured in the pressure cuff.

In order to further reduce the possibility of undesirable damage to the flexible vessel 14 when entrapped in the pressure cuff, the strap as illustrated in FIG. 2 is preferably transversely flared along its width direction to conform with the convex axial flare of the pad seat. Although the strap is flexible, it has hoop strength which creates reaction forces against the flexible vessel as it is entrapped. By suitably flaring or curling radially outwardly from the entrapped vessel the opposite width ends of the strap, a smoother transition from the circular profile of the vessel to the generally D-shaped profile of the vessel within the strap is obtained. The flared edges of the strap therefore reduce or eliminate any stress concentration at their junctions with the entrapped vessel.

The flexible strap 32 may be formed of any suitable biocompatible material including metal, plastic, or fibers, and is preferably a woven fabric as illustrated. The fabric may have suitable fibers of fiberglass, or polyester, or other material preferably coated with a friction reducing material such as polytetrafluoroethylene (PTFE) commonly known under the trademark TEFLON. This coated fabric is flexible and smooth and entraps the flexible vessel for minimizing abrasion thereof.

The use of a woven fabric strap 32 permits tailoring of its retention capabilities around the flexible vessel by the introduction of the end flares or curls on the opposite side edges of the strap in various manners. For example, the strap may include substantially rigid transverse fibers across its width having the desired convex profile with opposite curled ends as illustrated in FIG. 2, while still being flexible around its circumference in the form of a typical metal watch band.

Alternatively, FIG. 4 illustrates that the strap 32 has a center band 32a and forward and aft end bands 32b corresponding with the opposite edges along the width of the strap. The center and end bands may be differently woven so that the end bands are more flexible in the circumferential direction along the length of the strap than that of the center band. Either weaving, or the introduction of elastic fibers may be used to provide more flexibility for the end bands than for the center band to permit the ends bands to flare or curl in the manner illustrated in FIG. 2 for preventing localized stress concentration of the band as it entraps the flexible vessel.

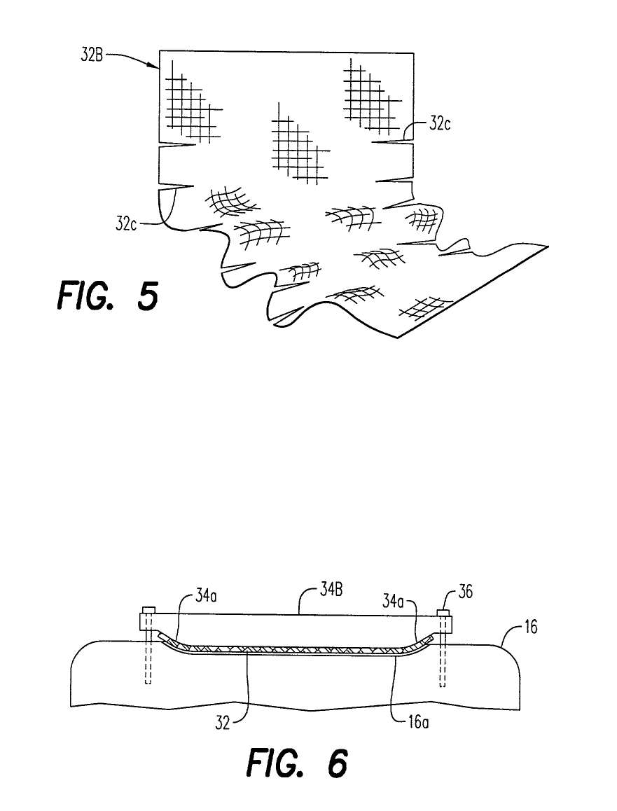

FIG. 5 illustrates an alternate embodiment of the flexible woven fabric strap, designated 32B, in which the two end bands on opposite sides of the strap width include small slits 32c extending inboard or inwardly from the opposite edges toward the center band. The slits are spaced apart from each other along each of the two opposite edges of the strap so that under tension load along the length of the strap the spacing between the slits enlarges to effectively curl or flare the edges of the strap as the strap entraps the flexible vessel. In this way, the edges of the strap prevent the introduction of localized stress concentration thereat for reducing damage or trauma to the entrapped vessel.

As shown in FIGS. 1 and 2, the clamp bars 34 are preferably axially straight along the strap to clamp the strap flat against the cooperating sides of the pad. FIG. 6 illustrates an alternate embodiment of the clamping bars, designated 34B, which is substantially straight yet includes arcuate opposite ends 34a to clamp the strap 32 against complementary arcuate recesses 16a in the sides of the pad. In this way, the clamping configuration may conform with the desired end-curling of the flexible strap 32 to ensure uniform contact between the strap and the entrapped vessel along the full width of the strap for spreading the retention loads uniformly therealong.

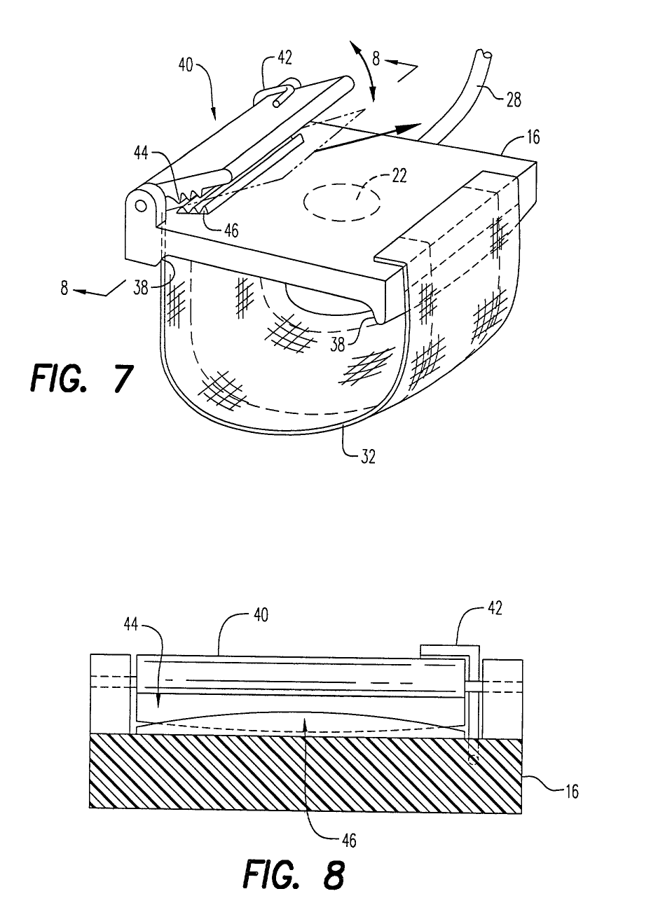

FIG. 7 illustrates an alternate embodiment of the pressure cuff in which the strap joining means comprise a ratchet lever 40 suitably pivotally joined to hinge extensions of the pad 16 for releasably clamping the corresponding end of the strap 32 thereagainst. A proximal end of the strap 32 may be fixedly joined to one end of the pad 16 in any suitable manner, such as by being permanently adhesively bonded thereto or using the clamp 34, 34B of FIGS. 1 & 6. The opposite distal or free end of the strap may then be threaded between the lever 40 and the pad at the opposite end thereof for conveniently installing the cuff, removing the cuff, and adjusting tension in the cuff strap.

The ratchet lever 40 preferably includes a spring 42 shown in different views in FIGS. 7 and 8 which is suitably configured for providing a closing force on the lever to pivot the lever downwardly against the pad. The lever includes a few rows of outer teeth 44, and the pad includes a few rows of complementary inner teeth 46 which engage each other for clamping the strap therebetween.

During installation, the free end of the strap, illustrated in phantom line in FIG. 7, is threaded between the outer and inner teeth while the ratchet lever is manually held upwardly for permitting free access therebetween. The strap is pulled through the lever slot for adjusting the effective length of the strap around the flexible vessel being entrapped. Releasing the lever permits the spring 42 to force the outer teeth against the inner teeth and frictionally trap the strap therebetween. The teeth prevent release of the strap until the lever is lifted upwardly.

As shown in FIG. 8, both the outer and inner teeth 44, 46 are straight along the length of the lever, but are preferably arcuate and taller centrally than at opposite ends thereof. In this way, as the teeth engage the flexible strap therebetween, the center of the strap is first engaged prior to the opposite edges of the strap which may be used for introducing the edge flares or curls in the strap when entrapping the flexible vessel.

The various embodiments disclosed above enjoy many benefits in measuring pressure in a flexible vessel without requiring any physical access to the fluid flowing therethrough. The pressure cuff is conveniently installed around the external surface of the flexible vessel and clamped thereagainst using the flexible strap. The flat diaphragm of the pressure sensor is thusly disposed in flat contact with the external surface of the vessel and effectively detects pressure of the internal fluid as transmitted through the vessel wall.

Although the pressure cuff may be used for measuring pressure in an artificial vessel, it also has particular utility in measuring pressure through an organic or living vessel such as an artery. Arterial walls are complex living structures and are subject to undesirable damage or trauma if not adequately protected.

The flexible strap of the pressure cuff disclosed above reduces the likelihood of disturbing the fluid balance within the arterial wall itself for minimizing trauma thereto. The arterial vessel is constrained or entrapped in most part by the conforming flexible strap, while minimizing the area of the rigid seat and corresponding flat diaphragm contacting the top of the arterial wall. This combination of elements also helps reduce or avoid undesirable trauma to the arterial wall.

The flexibility of the strap may be selected to match the flexibility of the specific form of the entrapped vessel for minimizing load concentration thereagainst. And, the side edges of the strap may be contoured or flared for reducing load concentration on the vessel, and may also be elastically stretchable for that purpose.

The preferred flexible strap therefore spreads the retention loads over a relatively large surface area of the arterial wall and minimizes local load concentration thereagainst to prevent restriction of the interstitial fluids within the arterial wall.

Fluid balance with the arterial wall is influenced by the pressure in the arterioles therein which drives fluid into the interstitial spaces in the wall, as well as by the lymph channels within the arterial wall which drain fluids from the interstitial spaces. By maximizing the flexible portions of the pressure cuff described above, the likelihood of local load concentration in the vessel wall is reduced for minimizing adverse effect on fluid balance within the arterial wall to reduce or avoid undesirable tissue swelling of the vessel.

The pressure cuff described above in various embodiments may be used in any application in which pressure measurement of a fluid inside a flexible container is desired. The fluid pressure is directly transmitted through the vessel wall to the flat diaphragm of the pressure sensor for accurate measurement thereof. The pressure cuff may be configured as desired for entrapping the vessel of any desired configuration with a flexible strap of complementary form.

The invention has particular utility in measuring pressure in an arterial graft through which blood is discharged from a LVAD used for assisting blood flow through a living heart.

The pressure cuff may be formed in various sizes and may be developed for minimizing the overall size thereof as suitable pressure sensors are correspondingly miniaturized in further developments. As the size of the pressure sensor itself is reduced, the corresponding size of the pressure cuff including the supporting pad may also be reduced for minimizing the area and applied loads surrounding the arterial vessel for which pressure measurement is desired.

In this way, the size of the supporting pad 16 may be minimized according to the state-of-art for reducing its constituent portion retained by the flexible retention strap. Maximizing the flexible portion of the cuff while minimizing the rigid portion thereof will correspondingly reduce undesirable damage or trauma to the arterial vessel, or other forms of living pressure vessels.

While there have been described herein what are considered to be preferred and exemplary embodiments of the present invention, other modifications of the invention shall be apparent to those skilled in the art from the teachings herein, and it is, therefore, desired to be secured in the appended claims all such modifications as fall within the true spirit and scope of the invention.

Accordingly, what is desired to be secured by Letters Patent of the United States is the invention as defined and differentiated in the following claims in which we claim:

1. A pressure cuff for measuring pressure in a flexible vessel comprising:
a pad having a seat for receiving said vessel therealong, and including an internal well;
a pressure sensor mounted in said well, and having a flexible flat diaphragm in said seat for engaging said vessel in flat contact therewith; and
a flexible strap adjustably joined to said pad for clamping said pad and seat in abutting contact with said vessel.

2. A cuff according to claim 1 further comprising means for joining opposite ends of said strap to said pad for defining a loop around a bottom side of said vessel, with an opposite top side of said vessel being compressible flat against said diaphragm.

3. A cuff according to claim 2 wherein said seat is axially flared convex between opposite axial ends of said pad.

4. A cuff according to claim 3 wherein said seat is circumferentially flared concave between opposite circumferential curbs of said pad.

5. A cuff according to claim 4 wherein said strap is axially flared to conform with said axial flare of said seat.

6. A cuff according to claim 5 wherein said pad is sized to surround a minority of circumference of said vessel, and said strap is sized to surround a majority of said vessel circumference.

7. A cuff according to claim 5 wherein said strap comprises a woven fabric.

8. A cuff according to claim 5 wherein said strap is axially wider than long between said pad curbs.

9. A cuff according to claim 5 wherein said strap comprises a center band, and forward and aft end bands being more flexible than said center band.

10. A cuff according to claim 5 wherein said strap comprises a center band, and forward and aft end bands having slits extending inwardly therein.

11. A cuff according to claim 5 wherein said strap joining means comprise a clamp fixedly joined to said pad for clamping said strap thereagainst.

12. A cuff according to claim 11 wherein said clamp comprises a bar straddling said strap against said pad, and a pair of fasteners at opposite ends thereof threadingly engaging said pad.

13. A cuff according to claim 12 wherein said clamp bar is straight along said strap to clamp said strap against said pad.

14. A cuff according to claim 12 wherein said clamp bar has arcuate opposite ends to clamp said strap against complementary arcuate recesses in said pad.

15. A cuff according to claim 5 wherein said strap joining means comprise a ratchet lever pivotally joined to said pad for releasably clamping said strap thereagainst.

16. A cuff according to claim 15 wherein said ratchet lever comprises rows outer teeth, and said pad includes complementary rows of inner teeth for clamping said strap therebetween.

17. A cuff according to claim 16 wherein said outer and inner teeth are taller centrally than at opposite ends thereof.

18. A cuff according to claim 5 wherein said pressure sensor includes a flat metal diaphragm covered by an elastomeric diaphragm being flush with said seat.

19. A cuff according to claim 18 wherein said seat is flat between said curbs.

20. A cuff according to claim 19 wherein said seat has a width generally equal to the diameter of said strap loop.
